(19) United States
(12) Patent Application Publication
(10) Pub. No.: US 2014/0134045 A1
(43) Pub. Date: May 15, 2014

(54) PROCESS FOR REDUCING MICROBIAL GROWTH IN CONTACT LENS STORAGE CASES

(71) Applicants: Jeremy Storer Chrisp, Hunters Hill (AU); Mark Duncan Perry Willcox, Balmain (AU); Ajay Kumar Vijay, Randwick (AU); Hua Zhu, Bexley (AU)

(72) Inventors: Jeremy Storer Chrisp, Hunters Hill (AU); Mark Duncan Perry Willcox, Balmain (AU); Ajay Kumar Vijay, Randwick (AU); Hua Zhu, Bexley (AU)

(73) Assignee: Warm Contact Pty Ltd., Mitchell (AU)

(21) Appl. No.: 14/113,889

(22) PCT Filed: Apr. 26, 2012
(86) PCT No.: PCT/AU2012/000433

§ 371 Date: Jan. 14, 2014

(30) Foreign Application Priority Data
Apr. 27, 2011 (AU) 2011901550

(51) Int. Cl. A61L 2/04

(52) U.S. Cl. CPC A61L 2/04
USPC 422/22

(57) ABSTRACT

A method of disinfecting a contact lens storage case having a lid, the method comprising the steps of occluding the cavity of the case with the lid; and actively drying the case by heating the case for about three hours at about 60 degrees Celsius.

### TECHNICAL FIELD OF THE INVENTION

The present invention relates generally to contact lenses, their associated storage cases and hygiene treatment regimes, and in particular, to a process for reducing microbial growth in contact lens storage cases (hereinafter referred to as contact lens storage cases, storage cases, or cases).

### BACKGROUND

Contact eye lenses (hereinafter referred to simply as lenses) are becoming increasingly widespread. This has been accompanied by a corresponding increase in the occurrence of microbial keratitis and other eye infections.

Inadequate case hygiene, has been identified as one factor associated with the noted increased risk of developing eye infections, and several approaches are currently used to improve case hygiene.

One such approach is to use disinfecting solutions to reduce the level of microbes and other foreign particles that adhere to the contact lenses. In another arrangement, contact cases are infused with silver ions to reduce contamination on the case surface. Another regime (see WO 2010/111744 A1) involves actively drying the case, the lid positioned in a clip adjacent to the case to prevent ingress of airborne dust, microbes and mould spores into the case during the drying process and during the subsequent storage period between uses of the case, to thereby reduce the growth of flora and fauna in the case. The term “actively drying” means drying the contact lens storage case by heating the case to a temperature that significantly reduces drying time of the case and associated lid, when compared to the case being passively dried at ambient temperature and humidity.

However, despite availability of the above arrangements, and advice that is routinely given to lens wearers by medical and eyewear professionals about how to care for their cases, studies have shown that between 76 to 92% of cases are contaminated to a degree that allows eye infection to occur.

### SUMMARY

It is an object of the present invention to substantially overcome, or at least ameliorate, one or more disadvantages of existing arrangements.

Disclosed are arrangements, referred to as occluded case drying (ie OCD) arrangements, which seek to address the above problems by occluding the cavity of the contact lens case with the case lid while actively drying the case.

The term “occluding” in the present specification includes within it's scope resting the lid on the case to thereby block or hide the cavity of the case, partially engaging the lid and the case, and fully engaging the lid and the case.

According to a first aspect of the present invention, there is provided a method of disinfecting a contact lens storage case having a lid, the method comprising the steps of occluding the cavity of the case with the lid, and then actively drying the case by heating the case for about three hours at about 60 degrees Celsius.

Other aspects of the invention are also disclosed.

### BRIEF DESCRIPTION OF THE DRAWINGS

At least one embodiment of the present invention will now be described with reference to the drawings and appendices, in which:

FIGS. 1A-1C shows a prior art apparatus for actively drying a lens case;

FIG. 2 shows an apparatus for disinfecting a contact lens storage case according to the disclosed OCD method;

FIG. 3 is a flow diagram of a method for disinfecting a contact lens storage case having a lid according to the present disclosure;

FIG. 4 is an artist's rendition of the OCD arrangement depicted in FIG. 2;

Appendix A is a report arising from a clinical study that the Applicant undertook to test the microbial contamination levels within storage cases under various conditions; and

Appendix B sets out a flow diagram of the clinical study protocol.

### DETAILED DESCRIPTION INCLUDING BEST MODE

Where reference is made in any one or more of the accompanying drawings to steps and/or features, which have the same reference numerals, those steps and/or features have for the purposes of this description the same function(s) or operation(s), unless the contrary intention appears.

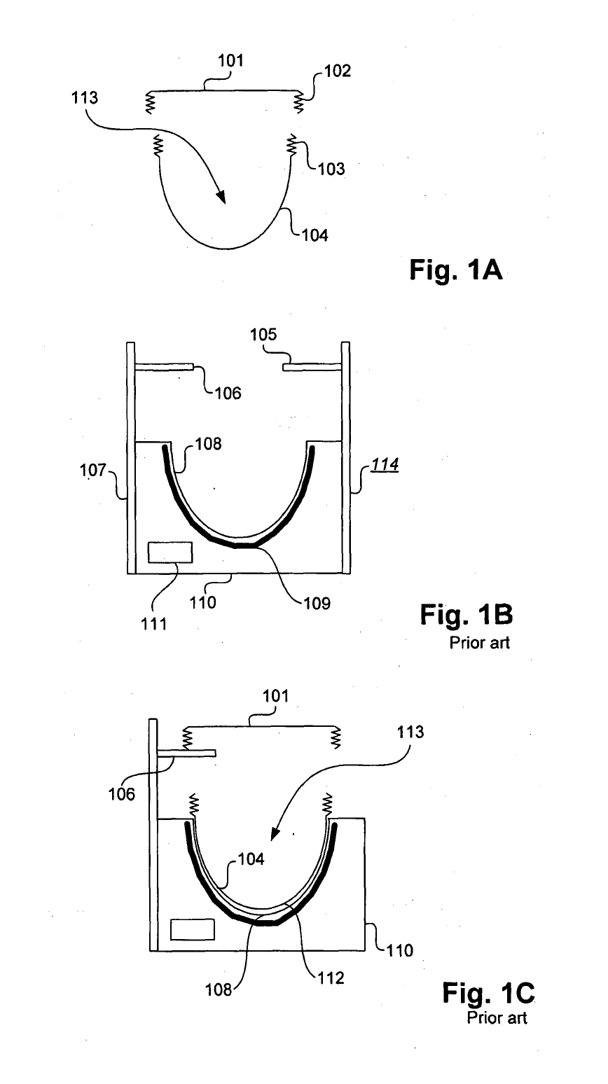

FIGS. 1A-1C shows a prior art apparatus for actively drying a lens case.

FIG. 1A depicts a contact lens storage case 104 and an associated lid 101. A contact lens (not shown) can be placed into a well (also referred to as a cavity) 113 in the case 104 when the lens is to be stored. The lid 101 has threads 102 that engage with threads 103 on the case 104 when the lid 101 is attached to the case 104 in order to close the case 104.

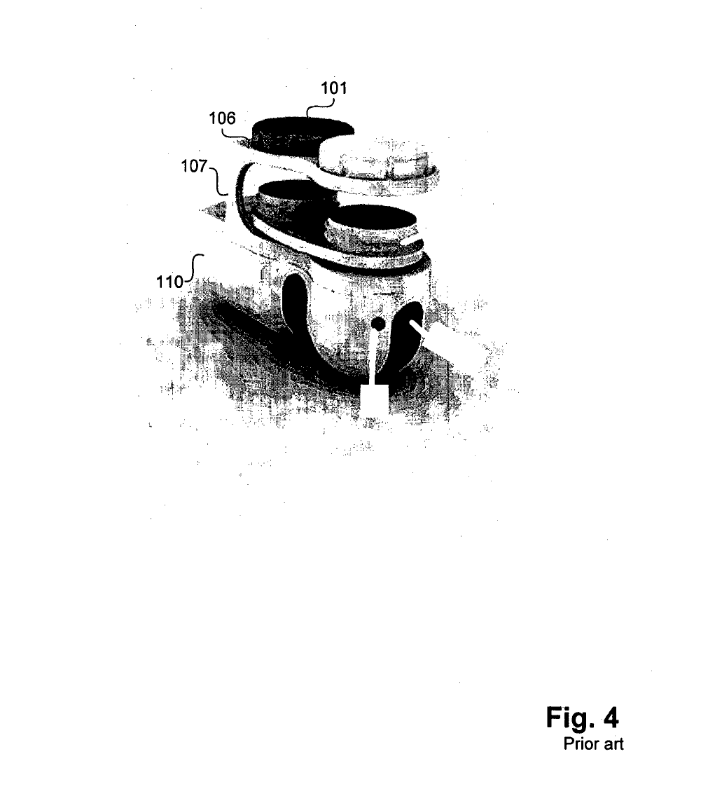

FIG. 1B depicts an apparatus 110 having a cavity 108 whose surface is conformal to an outer surface of the case 104. Accordingly, when a heating element 109 is activated by a power supply/control system 111 in the apparatus 110, a desired amount of heating energy can be precisely and repeatedly applied to the case 104 after the case 104 has been inserted into the cavity 108 in order to actively dry the case 104. Vertical members 107, 114 are attached to the sides of the apparatus 110, the vertical members 107, 114 having respective horizontal members 106 and 105 that act as an annular shelf for supporting the lid 101 of the case 104 as described in more detail in regard to FIGS. 1C and 4.

FIG. 1C depicts the apparatus 110 having the case 104 inserted into the cavity 108 of the apparatus 110. An edge of the lid 101 is supported on the annular shelf formed by the horizontal member 106, the other vertical member 114 and the associated horizontal member 105 having been omitted from FIG. 1C for clarity. This arrangement supports the lid 101 conveniently over the cavity 113 in the case 104, in order to allow the lid to dry by convection, and to shield the case 104 from ingress of airborne dust, microbes and mould spores.

FIG. 4 shows an artists rendition of the arrangement shown in FIG. 1C.

It is to be noted that the discussions contained in the “Background” section and the paragraph(s) above relating to prior art arrangements relate to discussions of documents or devices that may form public knowledge through their respective publication and/or use. Such discussions are not a representation by the present inventor(s) or the patent applicant that such documents or devices in any way form part of the common general knowledge in the art.

A clinical study has been undertaken by the Applicant to test the microbial contamination levels within storage cases that have been actively dried. The study included experiments to:
1. Determine the most effective operating temperature to heat the storage cases to in order to significantly reduce microbial activity;
2. Compare the performance of an active drying device (such as 110) to an incubating oven (not shown) operating at the same operating temperature;
3. Compare the biocidal effectiveness of actively drying the storage case when the lids were left off the container vs. when the lids were either resting on top of, partially or fully engaged with their respective screw threads;
4. Compare the biocidal effectiveness of actively drying the storage case against a control using commercially available multipurpose contact lens disinfection solution (MPDS); and
5. Determine the biocidal effectiveness of actively drying the storage case after following the multipurpose disinfection solution manufacturer recommended treatment regime.

Appendix B sets out a flow diagram of the clinical study protocol.

The results from the clinical study (the complete report is incorporated at APPENDIX A) determined the following:
1. In accordance with expectations, of the 3 temperatures tested (14° C., 45° C. and 60° C.), heating the storage cases to 60° C. for a period of 3 hours was the most effective temperature to significantly reduce microbial activity;
2. In accordance with expectations, while cases dried using the active drying device did not achieve the same very high level of efficacy as the incubating oven in reducing microbial activity they did significantly reduce microbial activity;
3. Unexpectedly drying the storage cases with the lids off was not as effective as drying the storage cases with the lids either resting on top of, partially or fully engaged with their respective screw threads;
4. As anticipated the active storage case drying device was more effective at reducing microbial activity than the commercially available multipurpose contact lens disinfection solution; and
5. Unexpectedly, combining both a manufacturer's recommended, commercially available, MPDS treatment regime and an active drying process resulted in significantly improved effectiveness in reducing microbial activity; the level of reduction of which was not anticipated.

Results 1, 2 and 4 largely met with expectations. Results 3 and 5 were surprising and totally unexpected.

More particularly, drying the storage cases with the lids on was significantly more effective than drying the storage cases and associated lids separately. Furthermore, the combination of the use of an MPDS regime and subsequent active drying resulted in considerably improved performance; reducing microbial activity to below measurable thresholds.

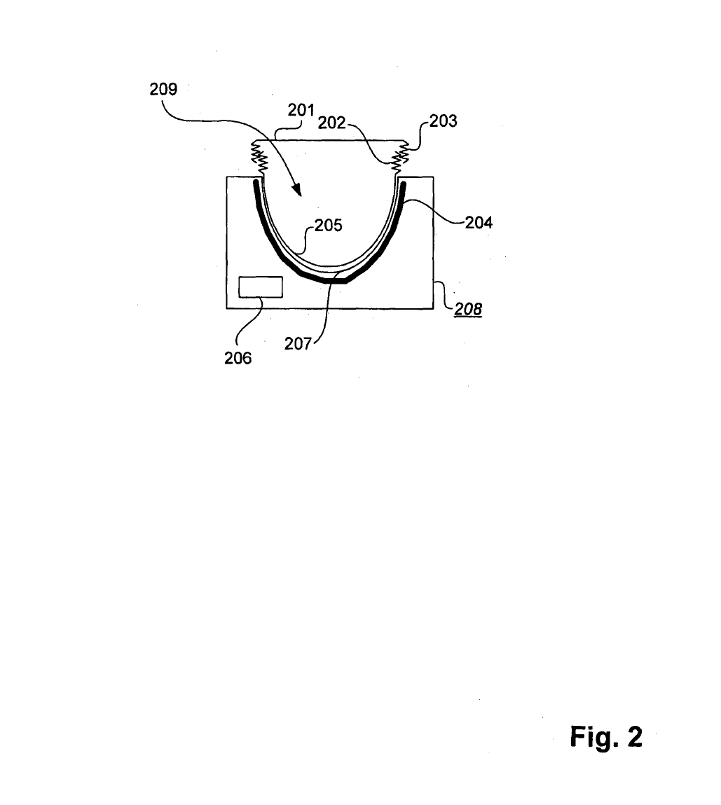

FIG. 2 shows an apparatus 208 for disinfecting a contact lens storage case according to the OCD method. The apparatus 208 is similar to the apparatus depicted in FIG. 1, however as explained below, there is a significant difference that enables the OCD method to be practiced. The apparatus 208 has a cavity 207 whose surface is conformal to an outer surface of a case 205. Accordingly, when a heating element 204 is activated by a power supply/control system 206 in the apparatus 208, a desired amount of heating energy can be precisely and repeatedly applied to a case 205 after the case 205 has been inserted into the cavity 207 in order to disinfect the case 205. In contrast to the apparatus 110 in FIG. 1B, there are no vertical members having horizontal members that acts as an annular shelf for holding a lid 201 of the case 205. Instead, the lid 201 is positioned in order to “occlude” a cavity 209 of the case 205 by either (a) resting the lid 201 on top of the case 205, or (b) partially or (c) fully engaging screw threads 203 of the lid 201 with corresponding screw threads 202 of the case 205.

It has been found, unexpectedly, that disinfecting the case 205 with the lid 201 occluding the cavity 209 of the case as described above greatly improves the efficacy of the MPDS, and results in reducing microbial activity to below measurable thresholds. Furthermore, disinfecting the case according to the OCD method after using an MPDS treatment regime significantly improves the antimicrobial effect of the MPDS on the case. More particularly, disinfecting the case by heating the case 205, with the lid 201 occluding the cavity of the case 205, for a period of about three hours at a temperature of about 60 degrees C. is highly effective. Other time periods and temperature settings can also be used for the OCD method.

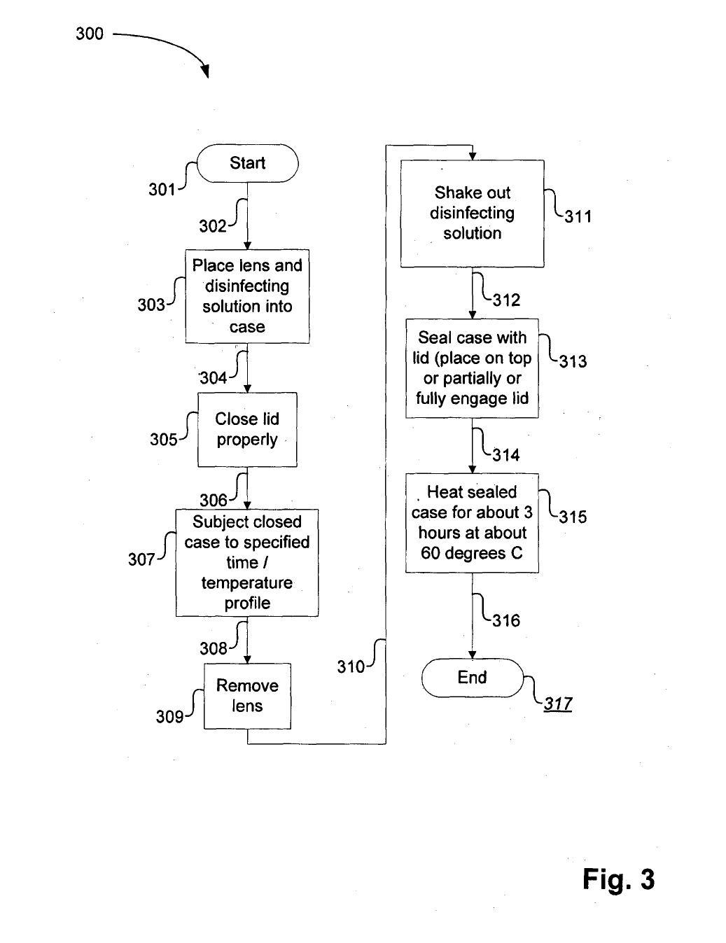

FIG. 3 is a flow diagram of a method 300 for disinfecting a contact lens storage case 205 having a lid 201, according to the disclosed OCD method. The method 300 commences with a start step 301 and then follows an arrow 302 to a step 303. In the step 303 the user places a contact lens into the cavity 209 of the case 205. In accordance with recommended procedures, a predetermined volume of MPDS is deposited in the cavity of the case in the step 303. The process 300 then follows an arrow 304 to a step 305. In the step 305 the user secures the lid 201 to the case 205 tightly. The process 300 then follows an arrow 306 to a step 307. In the step 307 the case, the contact lens in the case, the disinfecting solution and the lid are subjected to a time/temperature profile as specified by the manufacturer of the disinfecting solution. The process 300 then follows an arrow 308 to a step 309. In the step 309 the user removes the contact lens from the case. The process 300 then follows an arrow 310 to a step 311. In the step 311 the user shakes out the disinfecting solution. The user does not rinse out the case or manually dry the case. The process 300 then follows an arrow 312 to a step 313. In the step 313 the user occludes the cavity of the case by either resting the lid on top of the case, or partially or fully engaging the screw threads of the lid with their respective screw threads on the case. The process 300 then follows an arrow 314 to a step 315. In the step 315 the case is sterilized for a period of about 3 hours at about 60° C. The process 300 then follows an arrow 316 to a step 317 at which time the case is ready for reuse.

Appendix A is a report arising from the clinical study that the Applicant undertook to test the microbial contamination levels within storage cases under various conditions.

Appendix B sets out a flow diagram of the clinical study protocol.

### INDUSTRIAL APPLICABILITY

The arrangements described are applicable to the contact lens industry.

The foregoing describes only some embodiments of the present invention, and modifications and/or changes can be made thereto without departing from the scope and spirit of the invention, the embodiments being illustrative and not restrictive.

Accordingly, other temperature ranges and time intervals can be specified, in order to optimise the OCD process in particular circumstances. These circumstances may depend, among other considerations, upon the treatment regime specified for the MPDS, the active drying device utilised and so on. Furthermore, in order to occlude the cavity of the case, engagement of the case and the lid can be achieved using threads as described or other engagement methods such as complimentary press-fit surfaces.

1. A method of disinfecting a contact lens storage case having a lid, the method comprising the steps of:
occluding the cavity of the case with the lid; and
actively drying the case by heating the case for about three hours at about 60 degrees Celsius.

2. The method according to claim 1, wherein the step of occluding the cavity of the case comprises one of:
resting the lid on the case to thereby hide the cavity of the case;
partially engaging the lid and the case; and
fully engaging the lid and the case.

3. The method according to claim 2, where the step of partially engaging the lid and the case comprises partially engaging respective screw threads of the lid and the case.

4. The method according to claim 2, where the step of partially engaging the lid and the case comprises partially engaging respective press fit surfaces of the lid and the case.

5. The method according to claim 2, where the step of fully engaging the lid and the case comprises fully engaging respective screw threads of the lid and the case.

6. The method according to claim 2, where the step of fully engaging the lid and the case comprises fully engaging respective press fit surfaces of the lid and the case.

7. The method according to claim 1, wherein prior to the occluding and the actively drying steps the method comprises the further steps of:
depositing a contact lens and an amount of disinfecting solution into the cavity of the case;
fully engaging the lid and the case;
subjecting the case to a time/temperature regime specified for the disinfecting solution;
removing the contact lens; and
manually shaking out the disinfecting solution.

8. The method according to claim 1, wherein the actively drying step comprises actively drying the case by heating the case having the cavity occluded by the lid for between 1 and 4 hours at 60 degrees Celsius.

9. The method according to claim 1, wherein the actively drying step comprises actively drying the case by heating the case having the cavity occluded by the lid for between 1 and 4 hours at between 55 and 65 degrees Celsius.

10. The method according to claim 1, wherein the actively drying step comprises actively drying the case by heating the case having the cavity occluded by the lid for between 1 and 4 hours at between 50 and 70 degrees Celsius.

11. The method according to claim 1, wherein the actively drying step comprises actively drying the case by heating the case having the cavity occluded by the lid for between 1 and 4 hours at between 45 and 75 degrees Celsius.
